(19) United States
(12) Patent Application Publication
(10) Pub. No.: US 2021/0268159 A1
(43) Pub. Date: Sep. 2, 2021

(54) ELECTRICAL BREAST PUMP

(71) Applicant: MAM BABYARTIKEL GESELLSCHAFT M.B.H., Wien (AT)

(72) Inventors: Herfried SCHWARZ, Klosterneuburg (AT); Thomas ROHACZEK, Wien (AT); Vanessa RAMSTER, München (DE); Philipp PELZL, Rosenheim (DE); Philip ZEITLER, München (DE); Andreas BEER, München (DE); Peter RÖHRIG, Wien (AT)

(73) Assignee: MAM BABYARTIKEL GESELLSCHAFT M.B.H., Wien (AT)

(21) Appl. No.: 17/260,469

(22) PCT Filed: Jul. 17, 2019
(86) PCT No.: PCT/EP2019/069172

§ 371 Date: Jan. 14, 2021

(30) Foreign Application Priority Data
Jul. 17, 2018 (EP) 18183942.4

(51) Int. Cl. A61M 1/06; A61M 1/00

(52) U.S. Cl. CPC A61M 1/064; A61M 1/60; A61M 2205/8206; A61M 2205/587; A61M 2205/3337

(57) ABSTRACT

The invention relates to an electrical breast pump (12) comprising a breast cover (2) having a breast funnel (3) for at least partially receiving the breast, a pumping device (7) having a motor (8) and preferably one energy accumulator (10), and a milk storage unit (4) with having a milk container (5) for receiving the pumped milk, wherein, in a standing position of the electrical breast pump (1) on a surface (10), the breast cover (2) and the pumping device (7) lie substantially within a virtual general straight cylinder, wherein the base area thereof corresponds to the area—extended by a factor of 2.5, preferably by a factor of 2.0, particularly preferably by a factor of 1.8—of the greatest largest cross-section of the milk storage unit (4) parallel to the surface (10), wherein the area centroid of the base area is identical to the area centroid of the area of the greatest largest cross-section, and the height of the virtual general straight cylinder is continuous in both directions.

The object of protection relates to an electrical breast pump comprising a breast cover having a breast funnel for at least partially receiving the breast, a pumping device having a motor and preferably an energy storage device and a milk storage unit having a milk container for receiving the pumped milk.

A plurality of electrical breast pumps is known in the prior art. In this case, the pumping unit or the motor of the pumping unit can be accommodated either in the electrical breast pump itself or in a separate device.

For instance, US 2007/0060873 A1 discloses a milking pump comprising: a bottle; a milking pump head which is mounted to an opening of the bottle and has a milking cup which is flared out like a trumpet; and a negative pressure generating unit which is attached to the milking pump head in order to produce a negative pressure inside the milking cup. The milking pump is in this case held upright by a standing foot. Furthermore, the view of the milk flow in the embodiment with an emptying device mounted at the top is appreciably restricted.

The presently known electrical milk pumps therefore have disadvantages both when the motor or the pumping unit are integrated with the rest of the breast pump in one and also when they are separated. For example, in the breast pumps presently available in the prior art, it is necessary to fasten an additional standing foot to these so that they can stand stably. Furthermore, these breast pumps require external hoses or cable. Moreover, they only allow a very restricted view of the flow of pumped milk at best. Furthermore, it is not possible to illuminate a milk funnel satisfactorily. Furthermore, conventional breast pumps are very bulky which has a negative effect on storage, transport and use. A further substantial disadvantage of the electrical breast pumps of the prior art is that a (manual) holding similar to nursing is not possible with these during operation when pumping milk, in particular as a result of the shape of the milk funnel thereof, the angle and the distance of the funnel from a holding section of the breast pump and further unique features of the electrical breast pumps of the prior art. Investigations have shown that it would be seen as advantageous by mothers and midwives if the breast pump could be held in a (hand) position similar to that during nursing of a baby when pumping milk, which would instinctively be perceived as correct, pleasant and dignified and would make the pumping of the milk more pleasant and efficient, in particular also since the oxytocin required for the milk flow would be released with high probability/intensity—however this is not achieved by the electrical breast pumps of the prior art or is only achieved to a very limited extent. It is the aim of the present object of protection to eliminate or reduce at least one of these disadvantages.

This is achieved by an electrical breast pump as specified in the outset wherein in a standing position of the electrical breast pump on a surface the breast cover and the pumping device lie substantially within a virtual general straight cylinder, the base area whereof corresponds to the area of the largest cross-section of the milk storage unit parallel to the surface, stretched by a factor of 2.5, preferably by a factor of 2, particularly preferably by a factor of 1.8, wherein the area centroid of the base area is identical to the area centroid of the area of the largest cross-section and the height of the virtual general straight cylinder is infinite in both directions. It is thereby achieved in particular that the parts of the breast pump do not project too far away from the centre of gravity of the breast pump or beyond a surface on which the breast pump stands. Furthermore, since the pumping unit with the motor is provided inside this cylinder, the use of external cables/hoses is avoided. Furthermore, as a result a (manual) holding similar to nursing is possible during operation of the electrical breast pump for pumping milk since for this purpose in particular the breast funnel must not protrude too far since otherwise a natural (manual) holding similar to nursing is no longer required.

Stretching, where the area centroids of the original area and the image area are identical, is understood a homothetic transformation with the area centroid of the original area (and therefore also the image area) as the centre and the given factor as the stretching factor. The pumping device in particular comprises a pump for pumping milk from the breast. The milk storage unit in particular constitutes that part of the electrical breast pump which includes the milk container and substantially directly surrounds this (in its maximum filling state).

In a preferred embodiment the electrical breast pump has a housing which substantially comprises all the components of the breast pump, wherein the breast funnel forms an opening in the housing. Preferably also the entire housing is located in the virtual general straight cylinder specified above.

It is advantageous if the housing has a holding section which substantially has the shape of the lateral surface of a general cylinder or truncated cone having a substantially elliptical base area, wherein preferably an external height contour of the cylinder or truncated cone has an angle of between 30° and −30°, preferably between −15° and 15°, with respect to a plane defined by the circumference of the breast funnel and particularly preferably is substantially parallel to this. The height contour of the truncated cone is in this case in particular defined by a generatrix of the truncated cone on the side facing away from the breast funnel, in particular the generatrix of the lateral surface of the truncated cone furthest away from the circumference of the breast funnel. Likewise preferably a midpoint of the circumference of the breast funnel lies within a virtual general cylinder or truncated cone having infinite longitudinal extension, the base area whereof corresponds to the base area of the general cylinder or truncated cone mentioned above in this paragraph, stretched by a factor of 2.5, preferably by a factor of 2.0, particularly preferably by a factor of 1.5, even more preferably by a factor of 1.0, most preferably by a factor of 0.8, the lateral surface of which general cylinder or truncated cone corresponds to the shape of the holding section. As a result of this “rounded” holding section, in particular in combination as a result of the corresponding angle to the breast funnel and as a result of a small projection, or even offset inwards, of the breast funnel, in particular a particularly natural (manual) holding of the breast pump can be achieved when pumping milk.

Advantageously, the housing is designed to be substantially flush with the exception of the breast funnel. With the exception of the breast funnel is understood to mean that the housing is considered to be closed at the opening formed by the circumference of the breast funnel. Flush is understood to mean that no parts project from the electrical breast pump or its main body comprising the pumping unit. Thus, for example, the cleaning of the electrical breast pump can be simplified considerably. A flush exterior is also required for the stability of the breast pump since no parts protrude which could break off even under low loading. Furthermore as a result, a most natural possible (manual) holding of the breast pump is again made possible. In particular, also combined with the “rounded” holding section specified above, an ergonomics and a manual holding similar to the holding of a baby and a baby's head during nursing can also be achieved.

It is advantageous if the electrical breast pump and preferably the housing is/are designed in such a manner that the milk storage unit can be removed from the electrical breast pump. This makes it easier to empty the milk container or use the pumped milk. Furthermore, cleaning is simplified.

The milk storage unit can form the lowest part of the electrical breast pump in the standing position.

In a preferred embodiment of the electrical breast pump, said electrical breast pump, in particular the milk storage unit or preferably the housing of the breast pump, in particular the part of the housing which forms the shell of the milk storage unit, has a substantially flat standing area on which the electrical breast pump can stand in the standing position. The standing area can thus be formed in particular by the housing or by the milk storage unit. Preferably the breast pump can thus stand on the standing area without tipping without external support such as, for example, a standing foot or the like.

It is advantageous if the pumping device is arranged substantially between the breast cover and the milk storage unit, in particular in relation to a direction normal to the surface in the standing position of the breast pump on the surface. In this case, in the standing position of the breast pump the breast cover is preferably arranged substantially higher than the milk storage unit. In the standing position of the breast pump on a surface in relation to the direction normal to the surface the pumping device is preferably arranged between an uppermost point of the breast cover and a lowermost point of the milk storage unit, even more preferably between a midpoint of the breast funnel and an uppermost point of the milk container or the milk storage unit. This achieves a particularly space-saving arrangement of the pumping unit in the breast pump, with the result that the size and in particular the height of the breast pump can be reduced. Furthermore the centre of gravity is consequently displaced downwards with respect to a breast pump in which the pumping unit is arranged above the breast cover and the milk storage unit, with the result that an improved standing stability is obtained.

In a preferred embodiment, the pumping device, in particular the motor and/or the storage unit is arranged in the standing position substantially below a plane which is parallel to the surface and whose height above the surface corresponds to 3.0 times, preferably 2.5 times, particularly preferably 2.0 times the maximum extension of the milk storage unit parallel to the surface. As a result, the vertical stability of the breast pump is further enhanced due to a lowered centre of gravity.

It is advantageous if the breast cover has a hose section via which the breast funnel is connected to the milk container, preferably detachably. The hose section guides the pumped milk to the milk container. If the hose section is connected detachably to the milk container, for example, by releasing the connection, the milk container can advantageously be emptied through the opening of the milk container which had previously been connected to the hose section.

In a preferred embodiment, the hose section is firmly connected to the breast funnel or is formed integrally with this. Thus, the tightness of the system is improved, in particular with the integral configuration, manufacture is simplified and a space-saving arrangement is possible.

The breast cover can be removed from the electrical breast pump. The electrical breast pump or the housing of the electrical breast pump can have a flap. As a result of the flap, the correct fastening or connection of the breast cover to the remaining breast pump and optionally the hose section to the milk container is simplified.

In an advantageous embodiment the pumping device comprises a vacuum pump which is suitable for generating a vacuum in the hose section, wherein the hose section preferably has a valve, preferably a duck bill valve, in the direction of the milk container after an attachment point of the vacuum pump. Thus a negative pressure is built up in the breast cover including the hose section. During pumping milk thus collects on the duck bill valve which runs into the milk container as soon as a sufficient quantity of milk has been collected or the negative pressure in the hose section is temporarily reduced. Then, a three-way connection can be provided wherein, for example, the vacuum pump is firmly connected to a connector of this connection whereas the hose section and the milk container are detachably connected to respectively one connector of this connection.

In a preferred embodiment, the pumping device comprises a peristaltic pump (i.e. a peristaltic pump), wherein preferably the peristaltic pump generates the pumping power by mechanical action on the hose section. An advantage of using a peristaltic pump is that this automatically has an intermittent pump power during continuous operation with the result that the pumping behaviour approximates to the natural suckling of a baby automatically, i.e. without the pump needing to be controlled separately. Furthermore, this embodiment is advantageous with the arrangement of the pumping device substantially between breast funnel and milk container, wherein the hose section runs between these, since the pump can be arranged in a particularly space-saving manner (partially) in the rounding of the breast funnel and can act on the hose section thereof. Preferably rollers or sliding shoes of the peristaltic pump act on the hose section. Advantageously the hose section is supported on a side facing away from the peristaltic pump or the rollers/sliding shoes thereof or it has a greater stiffness on this side or is spanned over the rollers/sliding shoes, in particular in order to ensure the squeezing necessary for the pumping. The motor of the peristaltic pump can, for example, be inside or outside the circumference travelled by the rollers/sliding shoes.

In an advantageous embodiment of the breast pump, the pumping device comprises a vacuum pump which is suitable for generating a negative pressure in the milk container. Thus, a particularly space-saving arrangement can be achieved with the result that a smaller overall size of the breast pump is required. Preferably a detachable connection is provided between vacuum pump and milk container. In this embodiment it is advantageous if the milk container is rigid and firm so that it is not excessively contracted by the vacuum pump—as is possible in the case of a flexible milk container.

It is advantageous if the pumping device is decoupled from the milk-guiding parts of the breast pump, i.e. the milk funnel, the hose section or the milk container. For this purpose, a pot membrane is provided in the connection between pumping device and the respective milk-guiding part. It is advantageous if the pumping device is connected to a closed space which is in pressurized communication, preferably by means of a flexible membrane, in particular a pot membrane, with the breast funnel or the hose section or the milk container so that by periodically generating a negative pressure in the closed space a negative pressure can be periodically generated in the breast funnel or in the hose section or in the milk container (and therefore in the connected state ultimately in the breast funnel). Thus any contamination of the milk or any penetration of milk into the pumping device is effectively prevented.

In a preferred embodiment, the breast cover and preferably
a part of the housing surrounding the breast cover and/or a part of the housing surrounding the hose section or a portion of the hose section and/or a part of the housing surrounding the milk container and/or
the milk storage unit and/or
the milk container and/or
the hose section or a portion of the hose section
comprises a transparent material so that a milk flow is visible for a user of the electrical breast pump or in particular in the region of the breast funnel the correct application of the breast pump to the breast is facilitated. It can be desired by the user of the electrical breast pump to observe the milk flow. Furthermore, the user can thus estimate at any time the quantity of milk already pumped. Furthermore, the cleaning or the ensuring of a sufficient cleaning can be simplified as a result. In the normal operating mode of the breast pump for pumping milk, the transparent material is accordingly arranged in particular on the upper side and/or rear side, i.e. the side facing away from the breast funnel, of the breast pump in order to satisfy the viewing direction of the user of the breast pump. Furthermore, the correct position of the breast pump can be controlled.

The operating mode of the breast pump is that mode in which the breast cover is in contact with the breast of a user (in this disclosure used as equivalent to female user) to pump milk.

In an advantageous embodiment, the electrical breast pump has a light source to illuminate the breast cover. Thus, in particular the correct positioning of the breast cover for the operating mode of the breast pump is simplified. Furthermore, this can improve the user's view of the milk flow.

It is advantageous if the pumping device has a control circuit and preferably a user interface so that a user can operate the pumping device. The control circuit can in particular control the pumping device, for example, different pumping patterns can be executed. The user interface can, for example, have a button which can be operated by a user to switch the breast pump on and off. For example, several functions can be controlled with the one button, e.g. by means of pressing times of different length or by repeated pressing. A plurality of buttons can also be provided in order to set different modes etc. Furthermore, the user interface can comprise a device for the output of information, for example, LEDs or a touch (screen) in order, for example, to display charging state, milk flow, set mode etc. The user interface can also be set up via a (wireless) connection to another device, for example, a tablet, smart phone, PC, server or a remote control. Furthermore, it can also be possible that two or more electrical breast pumps can be controlled simultaneously via this (wireless) connection. This is in particular helpful when simultaneously pumping both breasts. A connection of two breast pumps can also be possible via this connection.

The breast pump can have an electrical connector. An energy storage device provided can be charged using this connector and/or the pumping device can be supplied directly with energy. The electrical connector can also be set up as a device for receiving wireless energy transmission; preferably the energy storage device comprises a lithium ion battery. Furthermore it can also be provided that two breast pumps can be connected via cable. In this case, only one of the two breast pumps can have the energy storage device and/or the control circuit and the other breast pump can be supplied with energy or controlled by the first breast pump. Also two breast pumps, for example, one of two interconnected breast pumps can be connected to a base device, on which the settings for the two breast pumps which are provided for the simultaneous application to respectively one breast are performed.

Advantageously, the pumping device is accommodated in a watertight manner, wherein preferably the housing is watertight. Thus, damage to the pumping device or the breast pump can be prevented and cleaning is simplified.

It is preferred if the milk container is designed as a flexible storage bag. Thus, the milk container can expand similarly to the supplied volume of pumped milk; there is little or no air in the path which must leave the milk container parallel to the pumped milk in order to release volume in the milk container for the milk.

In a preferred embodiment the milk storage unit has a fixed and rigid shell into which preferably the flexible storage bag is received which bag serves directly as milk container or is received into the one rigid milk container. In particular, when receiving the flexible storage bag this can be protected from damage as a result.

It is advantageous if a bottle body of a baby milk bottle is provided as milk container. This is preferably removable so that a baby can be easily fed with the pumped milk.

It is preferable if a circumference of the breast funnel lies substantially in one plane, wherein in the standing position of the electrical breast pump on the surface, the plane on the side on which the breast funnel is located encloses an angle between 60° and 110°, particularly preferably between 75° and 105°, with the surface. As a result, the breast pump is particularly handy to use since the alignment of the breast funnel corresponds to an ergonomic posture of the breast pump in the usual operating mode.

The invention will be illustrated hereinafter with reference to figures which show preferred embodiments and should not be interpreted as limiting for the scope of protection. The figures show in detail:

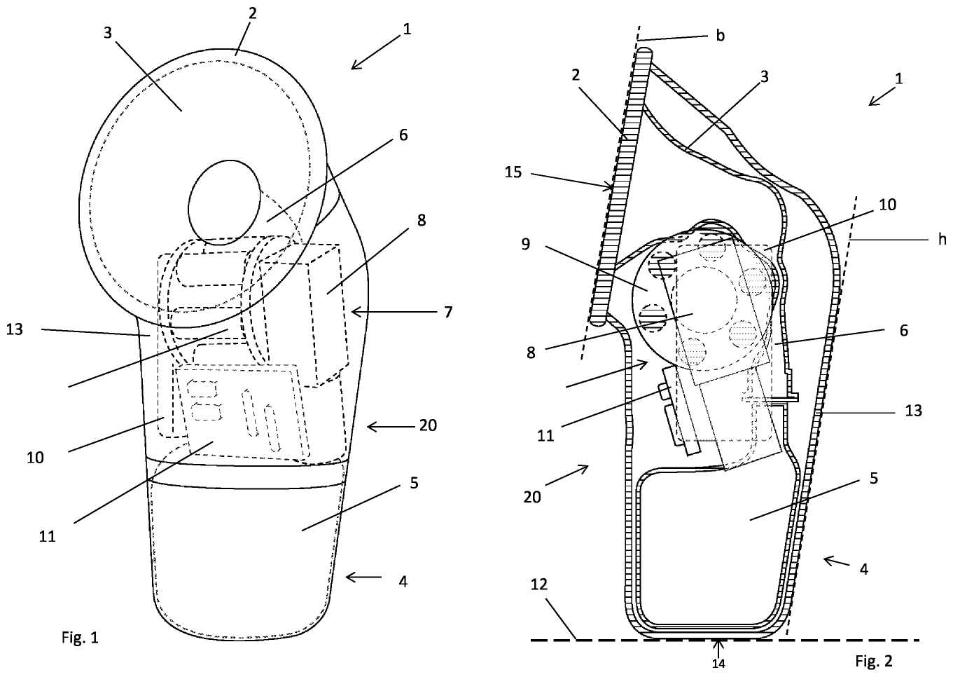

FIG. 1 shows an embodiment of the electrical breast pump with a peristaltic pump obliquely from the front;

FIG. 2 shows the same embodiment as FIG. 1 from the side;

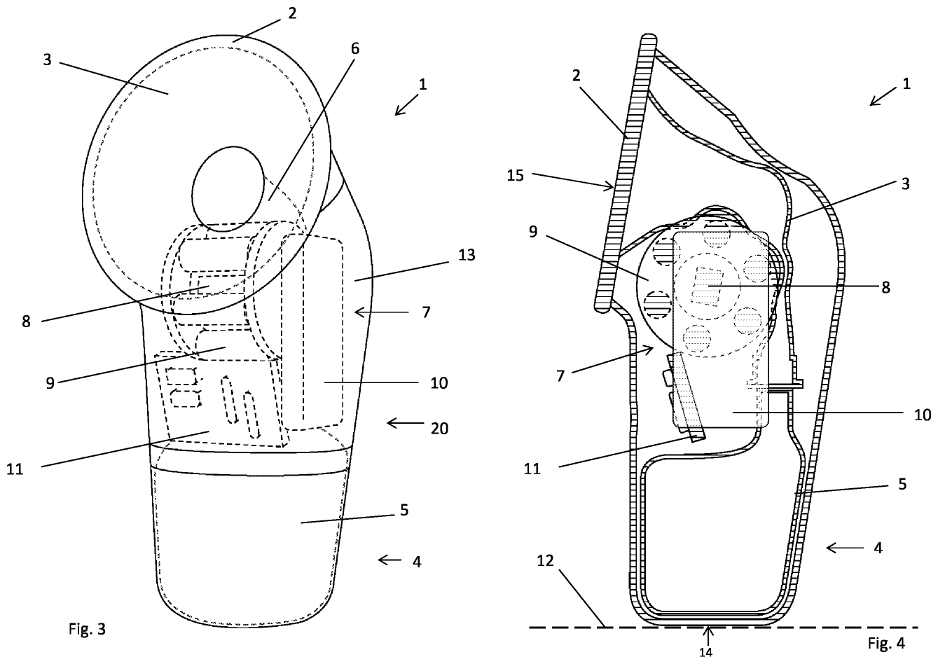

FIG. 3 shows a further embodiment of the electrical breast pump with a peristaltic pump obliquely from the front;

FIG. 4 shows the same embodiment as FIG. 3 from the side;

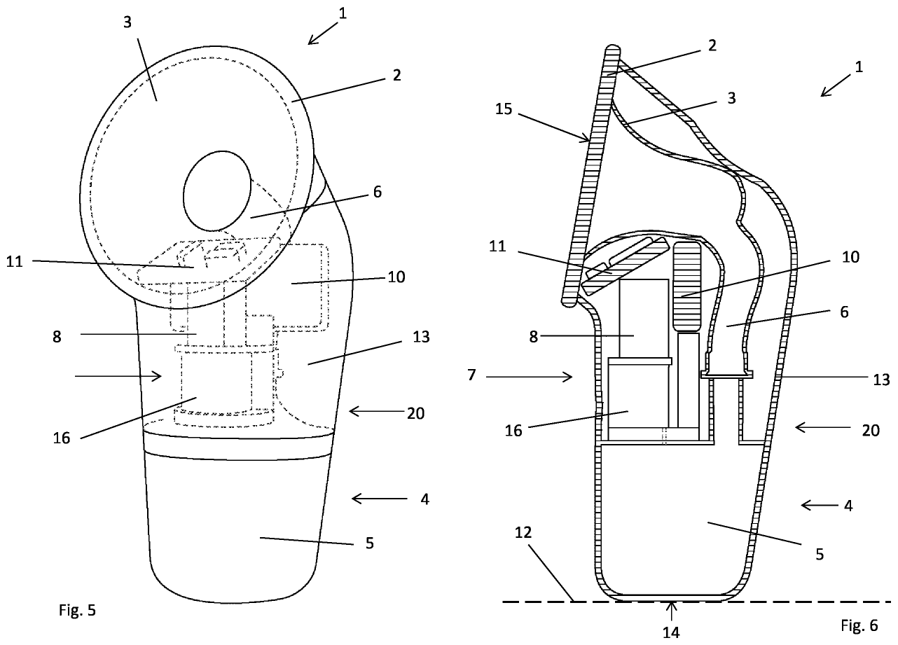

FIG. 5 shows an embodiment of the electrical breast pump wherein the vacuum pump is connected to the milk container, obliquely from the front

FIG. 6 shows the same embodiment as FIG. 5 from the side;

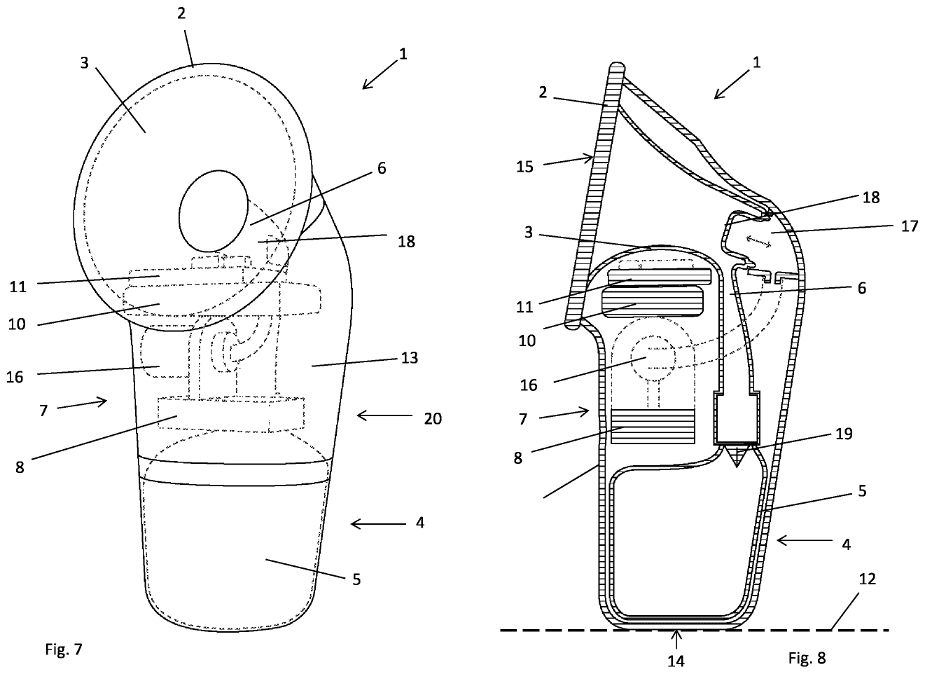

FIG. 7 shows an embodiment of the electrical breast pump wherein a vacuum pump is in pressurized communication with the breast funnel via a membrane, obliquely from the front;

FIG. 8 shows the same embodiment as FIG. 7 from the side;

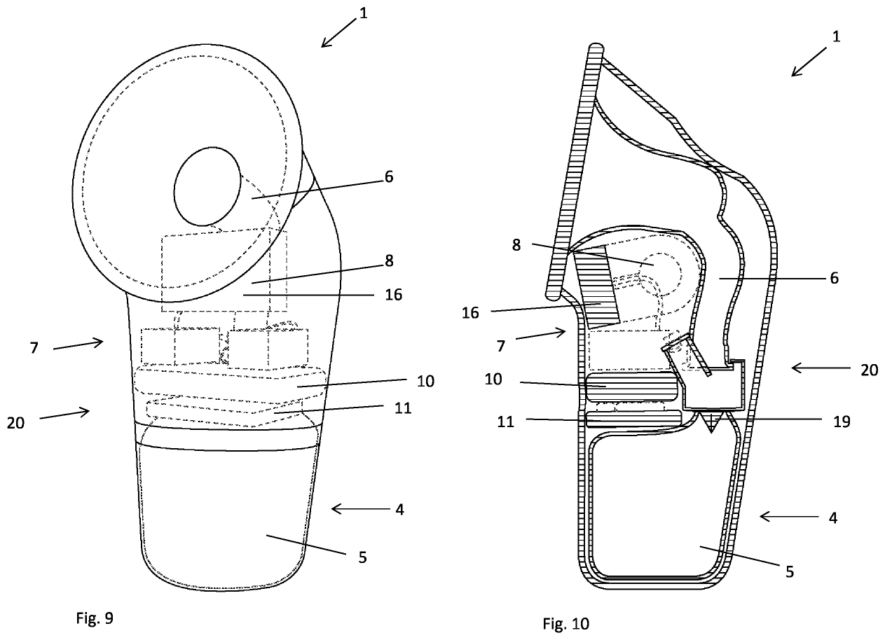

FIG. 9 shows an embodiment of the electrical breast pump wherein a vacuum pump is connected to the hose section or the milk container via a three-way connection, obliquely from the front;

FIG. 10 shows the same embodiment as FIG. 9 from the side;

FIG. 11 shows an embodiment of the electrical breast pump with peristaltic pump, wherein the milk storage unit can be removed, from the side;

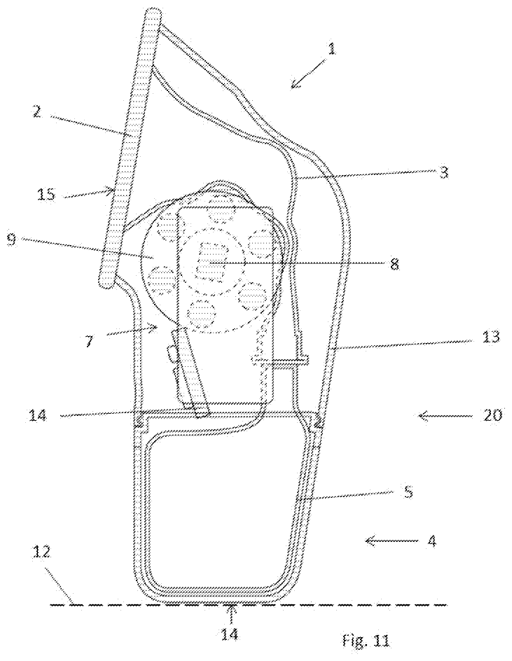

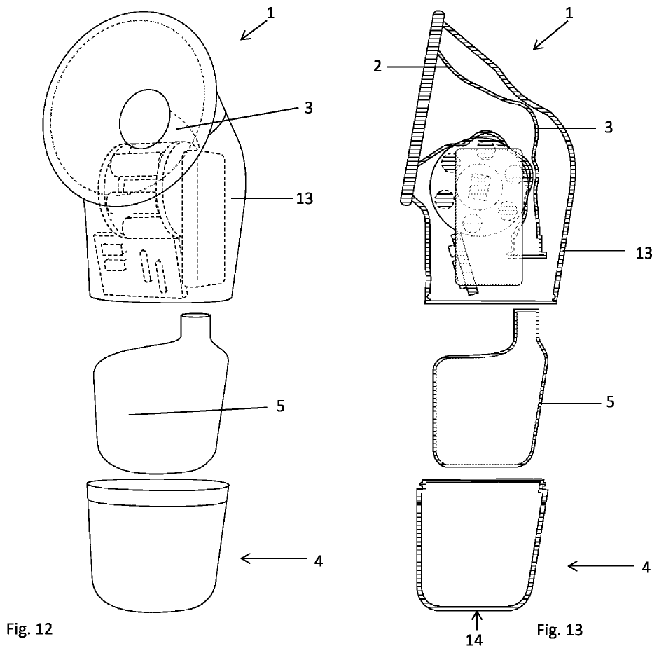

FIG. 12 shows the same embodiment as FIG. 11 obliquely from the side and with the milk storage unit removed;

FIG. 13 shows the same embodiment as FIG. 11 from the side and with the milk storage unit removed;

FIG. 14 shows an embodiment of the electrical breast pump with peristaltic pump, wherein the milk storage unit is executed as the bottle body of a baby milk bottle, obliquely from the side;

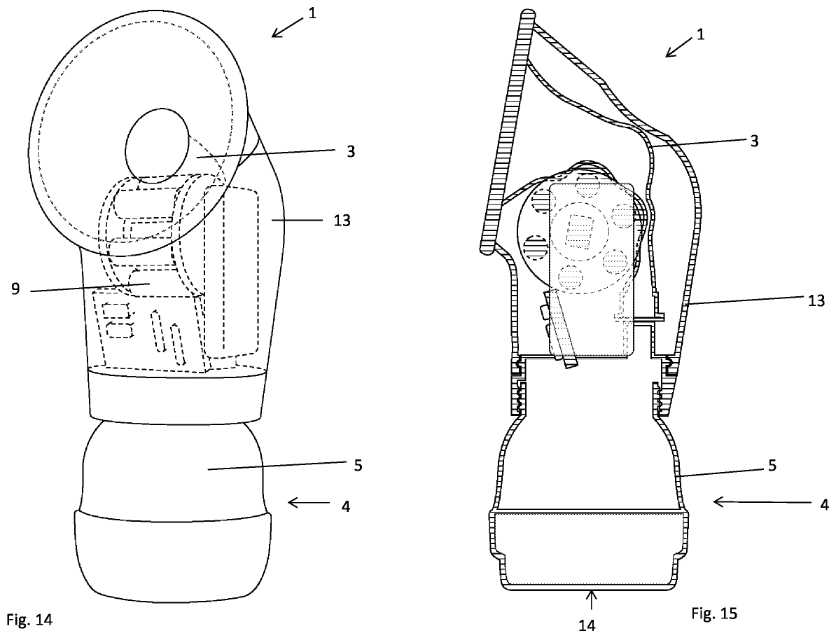

FIG. 15 shows the same embodiment as FIG. 14 from the side;

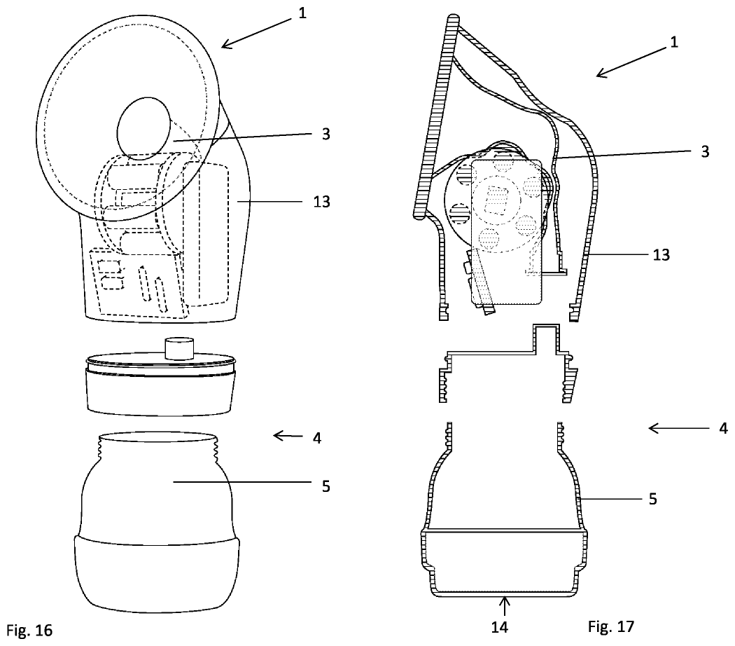

FIG. 16 shows the same embodiment as FIG. 14 with the milk storage unit removed, obliquely from the side;

FIG. 17 shows the same embodiment as FIG. 14 from the side and with the milk storage unit removed.

FIG. 1 shows an embodiment of the electrical breast pump 1 obliquely from the front, FIG. 2 shows the same embodiment in cross-section from the side. The breast pump 1 has a breast cover 2 with a breast funnel 3 for at least partially receiving the breast. Furthermore, said breast pump has a milk storage unit 4 with a milk container 5 for receiving the pumped milk. The breast cover 2 has a hose section 6 via which the breast funnel 3 is detachably connected to the milk container 5. Furthermore, the breast pump 1 comprises a pumping device 7 with a motor 8, a peristaltic pump 9, an energy storage device 10 and a control circuit 11. In this embodiment the motor 4 lies outside the peristaltic pump 9. The peristaltic pump 9 which is driven by the motor 8 acts on the hose section 6 and deforms it periodically mechanically so that in the operating mode milk/air is periodically pumped from the breast funnel 3 to the milk container 5. FIG. 2 shows the breast pump in the standing position on the surface 12.

The breast cover 2 and the pumping device 7 lie inside a general straight cylinder whose base area corresponds to the area of the largest cross-section of the milk storage unit 4 parallel to the surface 12 stretched by the factor 1.8, wherein the area centroid of the base area is identical to the area centroid of the largest cross-section and the height of the cylinder is infinite in both directions. In the standing position of the breast pump 1 on the surface 12 in relation to the direction normal to the surface 12 the pumping device 7 is arranged between a midpoint of the breast funnel 3 and an uppermost point of the milk container 5. In the standing position, the motor 8 and the energy storage device 10 are arranged substantially underneath a plane which is parallel to the surface 12 and the height of which above the surface 12 corresponds to 2.5 time the maximum extension of the milk storage unit 4 parallel to the surface 12. As a result of all these special arrangements, in particular the vertical stability of the breast pump 1 in the standing position can be improved.

The electrical breast pump 1 has a housing 13 which encompasses all the components of the breast pump 1, wherein the breast funnel 3 forms an opening in the housing 13. In this case, the housing 13 is designed to be substantially (with the exception of the breast funnel 3) flush. The housing 13 has a holding section 20 which substantially has the shape of the lateral surface of a general truncated cone with a substantially elliptical base area, wherein a height contour h of the truncated cone encloses an angle of about 15° with the plane b defined by the circumference 15 of the breast funnel 3. The height contour h is parallel to that generatrix of the truncated cone which can be seen on the right in FIG. 2 and thus lies on the side of the housing 13 facing away from the breast funnel 3.

It can be seen in FIG. 2 that the hose section 6 is detachable from the milk container 5. The milk storage unit 4 has a fixed and rigid shell which is part of the flush housing 13. In this embodiment in particular the milk container 5 can be a flexible storage bag (wherein it is shown here with its maximum volume). The hose section 6 is formed integrally with the breast funnel 3.

The breast pump 1 has a substantially flat standing area 14 on which it stands in the standing position. A circumference 15 of the breast funnel 3 lies in a plane which, in the standing position of the breast pump 1 on the surface 12, encloses an angle of around 80° with the surface 12.

As a result of the use of a peristaltic pump 9, the pumping device 7 can advantageously not come in contact with milk. The housing itself 13 is preferably also designed to be watertight.

FIG. 3 shows a similar embodiment of the breast pump 1 as in FIGS. 1 and 2 obliquely from the front; FIG. 4 shows the same embodiment as FIG. 3 in cross-section from the side. Unlike the embodiment of FIGS. 1 and 2, the motor 8 of the pumping device 7 is arranged inside the rollers of the peristaltic pump 9.

FIG. 5 shows an embodiment of the electrical breast pump 1 obliquely from the front, FIG. 6 shows the same embodiment in cross-section from the side. In this case, the pumping device 7 comprises a vacuum pump 16 which is connected to the milk container 5. In this case, the milk container 5 is advantageously rigid and firm. FIG. 7 shows an embodiment of the electrical breast pump 1 obliquely from the front, FIG. 8 shows the same embodiment in cross-section from the side. In this embodiment the pumping device 7 comprises a vacuum pump 16 which is connected to a closed space 17 which is in pressurized communication with the breast funnel 3 via a flexible membrane 18, a pot membrane. Thus, by periodically generating a negative pressure in the closed space 17 by the vacuum pump 16, the flexible membrane 18 can be moved periodically and thus a negative pressure in the breast funnel 3 can be generated periodically. In this embodiment as in the embodiments of FIGS. 1 to 4 it is also advantageous that the pumping device 7 cannot come in contact with the milk. Furthermore, the breast pump 1 has a duck bill valve 19 via which the milk container 5 is connected to the hose section 6. Thus, during pumping a negative pressure is built up in the breast cover 3 including the hose section 6 so that milk collects on the duck bill valve 19. As soon as a sufficient quantity of milk has been collected or as a result of the periodic removal of the vacuum, the duck bill valve 19 opens and the collected milk runs into the milk container 5. The milk container 5 can be a flexible storage container.

FIG. 9 shows an embodiment of the electrical breast pump 1 obliquely from the front, FIG. 10 shows the same embodiment in cross-section from the side. In this embodiment, the pumping device 7 comprises a vacuum pump 8. The vacuum pump 8 is connected to a three-way connection which in turn is connected to the hose section 6 and via a duck bill valve 19 to the milk container 5.

FIG. 11 shows an embodiment of the electrical breast pump 1 with removable milk storage unit 4 in a cross-section from the side; FIG. 12 shows the same embodiment as FIG. 11 obliquely from the side and with the milk storage unit 4 removed and FIG. 13 shows the same view as FIG. 11 with the milk storage unit 4 removed. This embodiment is similar to that of FIGS. 3 and 4 wherein in contrast to that embodiment, in this embodiment the milk storage unit 4 with the milk container 5 are removable from the rest of the breast pump 1. In FIG. 11 the milk storage unit 4 is connected to the rest of the breast pump 1. The housing 13 is flush in the fastened state of the milk storage unit 4. As can be seen, the milk storage unit 4 is connected to the rest of the housing 13. The milk container 5 is also detachably connected to the hose section 3. In FIGS. 12 and 13 the milk storage unit 4 is removed from the rest of the housing 13. Furthermore, the milk container 5 is also removed from the milk storage unit 4.

FIG. 14 shows an embodiment of the electrical breast pump 1 with the bottle body of a baby milk bottle as a removable storage unit 4 obliquely from the side; FIG. 15 shows the same embodiment in a cross-section from the side; FIGS. 15 and 17 show similar views as in FIGS. 14 and 15 with the milk storage unit 4 removed. This embodiment is similar to the embodiment of FIGS. 3 and 4 and also provides a peristaltic pump 9. In contrast to that however, a removable bottle body of a baby milk body is provided here as milk storage unit 4. The hose section 3 is detachably connected to an opening of the bottle body. In this embodiment the bottle body is in two parts, wherein an upper portion of the bottle body which also comprises the lid of the bottle body has a snap-in device for snapping in with the remaining housing 13. Furthermore, this upper portion has an internal thread into which an external thread of a lower portion of the bottle body can be screwed. In this embodiment, the bottle body has the standing area 14. It is also possible that the bottle body is formed in such a manner that the housing 13 is flush in the fastened state of the bottle body, wherein in the fastened state the bottle body forms a part of the housing 13.

1. An electrical breast pump comprising a breast cover having a breast funnel for at least partially receiving the breast, a pumping device having a motor and preferably an energy storage device and a milk storage unit having a milk container for receiving the pumped milk, wherein in a standing position of the electrical breast pump on a surface the breast cover and the pumping device lie substantially within a virtual general straight cylinder, the base area whereof corresponds to the area of the largest cross-section of the milk storage unit parallel to the surface stretched by a factor of 2.5, preferably by a factor of 2.0, particularly preferably by a factor of 1.8, wherein the area centroid of the base area is identical to the area centroid of the area of the largest cross-section and the height of the virtual general straight cylinder is infinite in both directions.

2. The electrical breast pump according to claim 1, wherein the electrical breast pump has a housing which substantially comprises all the components of the breast pump, wherein the breast funnel forms an opening in the housing.

3. The electrical breast pump according to claim 2, wherein the housing has a holding section which substantially has the shape of the lateral surface of a general cylinder or truncated cone having a substantially elliptical base area, wherein preferably
an external height contour of the cylinder or truncated cone encloses an angle of less than 30°, preferably less than 15°, with a plane defined by the circumference of the breast funnel and particularly preferably is substantially parallel to this and/or
a midpoint of the circumference of the breast funnel lies within a virtual general cylinder or truncated cone having infinite height, the base area whereof corresponds to the base area of the general cylinder or truncated cone stretched by a factor of 2.5, preferably by a factor of 2.0, particularly preferably by a factor of 1.5, even more preferably by a factor of 1.0, most preferably by a factor of 0.8, the lateral surface of which general cylinder or truncated cone corresponds to the shape of the holding section.

4. The electrical breast pump according to claim 2, wherein the housing is designed to be substantially flush, with the exception of the breast funnel.

5. The electrical breast pump according to claim 1, wherein the electrical breast pump and preferably the housing is designed in such a manner that the milk storage unit can be removed from the electrical breast pump.

6. The electrical breast pump according to claim 1, wherein the electrical breast pump, preferably the housing, in particular the milk storage unit, has a preferably substantially flat standing area on which the electrical breast pump can stand in the standing position.

7. The electrical breast pump according to claim 1, wherein the pumping device is arranged substantially between the breast cover and the milk storage unit.

8. The electrical breast pump according to claim 1, wherein the pumping device, in particular the motor and/or the energy storage device, is arranged in the standing position substantially below a plane which is parallel to the surface and whose height above the surface corresponds to 3.0 times, preferably 2.5 times, particularly preferably 2.0 times the maximum extension of the milk storage unit parallel to the surface.

9. The electrical breast pump according to claim 1, wherein the breast cover has a hose section via which the breast funnel is connected to the milk container, preferably detachably, wherein preferably the hose section is firmly connected to the breast funnel or is formed integrally with this.

10. The electrical breast pump according to claim 8, wherein the pumping device comprises a vacuum pump which is suitable for generating a negative pressure in the hose section, wherein the hose section preferably has a valve, preferably a duck bill valve, in the direction of the milk container after an attachment point of the vacuum pump.

11. The electrical breast pump according to claim 1, wherein the pumping device comprises a peristaltic pump, wherein preferably the peristaltic pump generates the pumping power by mechanical action on the hose section.

12. The electrical breast pump according to claim 1, wherein the pumping device comprises a vacuum pump which is suitable for generating a negative pressure in the milk container.

13. The electrical breast pump according to claim 1, wherein the pumping device is connected to a closed space which is in pressurized communication, preferably by a flexible membrane, with the breast funnel or the hose section or the milk container so that by periodically generating a negative pressure in the closed space a vacuum can be periodically generated in the breast funnel or in the hose section.

14. The electrical breast pump according to claim 1, wherein
the breast cover and preferably
a part of the housing surrounding the breast cover and/or a part of the housing surrounding the hose section or a portion of the hose section and/or a part of the housing surrounding the milk container and/or
the milk storage unit and/or
the milk container and/or
the hose section or a portion of the hose section
comprises a transparent material so that a milk flow is visible for a user of the electrical breast pump and/or
the electrical breast pump comprises a light source to illuminate the breast cover and/or
the pumping device comprises a control circuit and preferably a user interface so that a user can operate the pumping device and/or
the energy storage device comprises a lithium ion battery and/or
the pumping device is accommodated in a watertight manner, wherein preferably the housing is watertight and/or
the milk container is designed as a flexible storage bag and/or
the milk storage unit has a fixed and rigid shell into which preferably the flexible storage bag is received and/or
a bottle body of a baby milk bottle is provided as milk container of the milk storage unit (4).

15. The electrical breast pump according to claim 1, wherein a circumference of the breast funnel lies substantially in one plane, wherein in the standing position of the electrical breast pump on the surface, the plane on the side on which the breast funnel is located encloses an angle between 60° and 110°, particularly preferably between 75° and 105°, with the surface.
